(19) United States
(12) Patent Application Publication
(10) Pub. No.: US 2024/0036041 A1
(43) Pub. Date: Feb. 1, 2024

(54) KOKUMI TASTE ENHANCEMENT AND RELATED SCREENING METHODS

(71) Applicant: Firmenich Incorporated, Plainsboro, NJ (US)

(72) Inventors: Guy Servant, San Diego, CA (US); Eric Frerot, Satigny (CH)

(21) Appl. No.: 18/254,146

(22) PCT Filed: Nov. 19, 2021
(86) PCT No.: PCT/US2021/072519

§ 371 Date: May 23, 2023

Related U.S. Application Data
(60) Provisional application No. 63/117,775, filed on Nov. 24, 2020.

(51) Int. Cl. G01N 33/566; A23L 27/00; A23L 27/21; A23J 3/22; A23L 5/20

(52) U.S. Cl. CPC G01N 33/566; A23L 27/88; A23L 27/21; A23J 3/227; A23L 5/27; G01N 2333/726

(57) ABSTRACT

The present disclosure generally provides methods for identifying compounds that enhance kokumi taste, and related uses. In some particular aspects, the disclosure provides methods of identifying compounds that enhance kokumi taste by determining whether the compounds modulate the T1R1/T1R3 taste receptor. In some other aspects, the disclosure provides uses of such identified compounds to enhance the kokumi taste of an ingestible composition. In some further aspects, the disclosure provides flavored products, such as food or beverage products, that incorporate such ingestible compositions, and which thereby impart or enhance an umami taste in a flavored article. In some other aspects, the disclosure provides ingestible compositions that contain compounds known to impart or enhance umami taste, such as glutamates, aspartates, and purinic ribonucleotides, such as inosine monophosphate (IMP) and guanosine monophosphate (GMP). In some aspects, the inclusion of kokumi-enhancing compounds in flavored products allows one to reduce or eliminate the concentration of glutamate or aspartate in certain food and beverage products.

### CROSS-REFERENCE TO RELATED APPLICATIONS

The present application claims the benefit of priority of U.S. Provisional Application No. 63/117,775, filed Nov. 24, 2020, which is hereby incorporated by reference as though set forth herein in its entirety.

### TECHNICAL FIELD

The present disclosure generally provides methods for identifying compounds that enhance kokumi taste, and related uses. In some particular aspects, the disclosure provides methods of identifying compounds that enhance kokumi taste by determining whether the compounds modulate the T1R1/T1R3 taste receptor. In some other aspects, the disclosure provides uses of such identified compounds to enhance the kokumi taste of an ingestible composition. In some further aspects, the disclosure provides flavored products, such as food or beverage products, that incorporate such ingestible compositions, and which thereby impart or enhance an umami taste in a flavored article. In some other aspects, the disclosure provides ingestible compositions that contain compounds known to impart or enhance umami taste, such as glutamates, aspartates, and purinic ribonucleotides, such as inosine monophosphate (IMP) and guanosine monophosphate (GMP). In some aspects, the inclusion of kokumi-enhancing compounds in flavored products allows one to reduce or eliminate the concentration of glutamate or aspartate in certain food and beverage products.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have several basic taste modalities: sweet, bitter, sour, salty, and umami, and kokumi.

Kokumi is a word taken from the Japanese language to refer to the sensation of richness or deliciousness associated with many foods. In one early study, garlic extract was diluted to a concentration below its taste threshold, and combined with dilute solutions of glutamate and inosinate (compounds associated with umami taste). The resulting mixture had a rich garlic-like taste with a mouthfeel characteristic of the kokumi sensation. Through further work, it was determined that the kokumi sensation was associated with glutathione. Further studies have connected kokumi sensation with other glutamate-containing dipeptides and tripeptides, cysteine derivatives, histidine, and protamine. Examples of gamma-glutamyl tripeptides that enhance kokumi sensation include Glu-Glu-Leu and Glu-Val-Gly, and impart a taste sensation similar to that of glutathione.

Compounds that induce the kokumi taste sensation are present in a number of ingestible products, such as meats, mushrooms, and certain plants. But meats are probably the most typical sources of such compounds. Moreover, in foods that lack this sensation, one may try to enhance its absence through the inclusion of meat broths, salt, or compounds, such as monosodium glutamate (MSG), that impart umami taste. The inclusion of such additives in foods can have the effect of making the foods less healthy and more likely to contribute to adverse health consequences, such as hypertension, heart disease, and osteoporosis.

Enhancement of kokumi provides an alternative approach to partially or completely replacing ingredients that are traditionally used to impart kokumi taste. Until now, it has been widely believed that the calcium sensing receptor (CaSR) is responsible for mediating kokumi taste. Therefore, efforts to discover new kokumi-enhancing compounds have focused on screening against this receptor. But these efforts have thus far failed to yield a wealth of alternative compounds that can enhance the kokumi taste sensation. Thus, there is a continuing need to discover compounds that have improved ability to mimic the natural tastes of foods that traditionally exhibit a pleasurable kokumi taste.

### SUMMARY

The present disclosure relates to the discovery that kokumi taste is also mediated by the T1R1/T1R3 taste receptor, which is typically associated with the enhancement of umami taste. Thus, the methods disclosed herein provide a means of screening for compounds that may more closely approach the kokumi taste sensation typically imparted by glutathione and other peptides in meat and fungi.

In a first aspect, the disclosure provides methods of identifying compounds that produce or enhance a kokumi taste sensation, the method comprising: (a) introducing a test compound to a taste receptor, which comprises a first subunit and a second subunit; wherein the first subunit comprises a polypeptide sequence of SEQ ID NO: 1, a functional fragment thereof, or a polypeptide sequence whose sequence is at least 90% equivalent to either of the foregoing; and wherein the second subunit comprises (i) a polypeptide sequence of SEQ ID NO: 2, a functional fragment thereof, or a polypeptide sequence whose sequence is at least 90% equivalent to either of the foregoing, or (ii) a polypeptide sequence of SEQ ID NO: 3, a functional fragment thereof, or a polypeptide sequence whose sequence is at least 90% equivalent to either of the foregoing; and (b) measuring a response of the taste receptor to the test compound by comparing an activity of the taste receptor in the presence and the absence of the test compound. In some embodiments, the methods further comprise: identifying an active test compound that imparts or enhances kokumi taste sensation based on the measured response; and, optionally, selecting the active test compound as a compound that imparts or enhances kokumi taste sensation.

In a second aspect, the disclosure provides methods of identifying compounds that enhance a kokumi taste sensation, the method comprising: (a) introducing a test compound in the presence of a kokumi compound to a taste receptor, which comprises a first subunit and a second subunit; wherein the first subunit comprises a polypeptide sequence of SEQ ID NO: 1, a functional fragment thereof, or a polypeptide sequence whose sequence is at least 90% equivalent to either of the foregoing; and wherein the second subunit comprises (i) a polypeptide sequence of SEQ ID NO: 2, a functional fragment thereof, or a polypeptide sequence whose sequence is at least 90% equivalent to either of the foregoing, or (ii) a polypeptide sequence of SEQ ID NO: 3, a functional fragment thereof, or a polypeptide sequence whose sequence is at least 90% equivalent to either of the foregoing; and (b) measuring a response of the taste receptor to the test compound by comparing an activity of the taste receptor in the presence and the absence of the test compound.

In a third aspect, the disclosure provides uses of an identified or selected active test compound of any embodiments of the first or second aspect to impart of enhance kokumi taste sensation in an ingestible composition.

In a fourth aspect, the disclosure provides methods of imparting or enhancing kokumi taste sensation in an ingestible composition, the method comprising introducing to an ingestible composition an identified or selected active test compounds of any embodiments of the first or second aspect.

In an fifth aspect, the disclosure provides ingestible compositions comprising an identified or selected active test compounds of any embodiments of the first or second aspect. In some embodiments, the ingestible composition is a naturally occurring composition. In some embodiments, the ingestible composition is a non-naturally occurring composition.

In a sixth aspect, the disclosure provides flavored products comprising an ingestible composition of the fifth aspect. In some embodiments, the flavored products are food products or beverage products, such as savory foods (e.g., chips, crisps, etc.), beverages, soups, cheeses, snack foods, and any low-sodium foods. In some embodiments, the flavored product is a meat analogue product.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows the activity of certain gamma-glutamyl peptides, both in the presence and absence of inosine 5′-monophosphate (IMP), with respect to the T1R1/T1R3 taste receptor.

FIG. 2 shows the activity of certain gamma-glutamyl peptides with respect to cells not expressing the T1R1/T1R3 taste receptor.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

A “kokumi compound” refers to a compound that elicits a detectable kokumi taste sensation in a subject, such as the sensation produced by glutathione, Glu-Glu-Leu or Glu-Val-Gly. A “kokumi enhancer” refers to a compound that increases the activity of a kokumi compound, such as a compound that enhance the activity of glutathione, such as IMP or 5′-ribonucleotides.

A “functional fragment” refers to a portion of a polypeptide sequence to which the umami compound or the test compound binds. Polypeptide sequences often contain certain amino acids that do not actively participate in binding, but which may serve other purposes. In some instances, these non-functioning parts of the polypeptide sequence can be removed or partially replaced, while leaving the functional portion of the sequence intact. These modified proteins are said to comprise a functional fragment of the original polypeptide sequence.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Other terms are defined in other portions of this description, even though not included in this subsection.

### Polypeptide Sequences of Taste Receptors

The present disclosure provides certain polypeptide sequences that are useful in the simulating in vitro the response that certain human taste receptors would exhibit towards such compounds when ingested orally.

In some embodiments, the disclosure provides a polypeptide sequence of SEQ ID NO: 1, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 1 is set forth among the sequence listings filed herewith and incorporated by reference. For purposes of clarity and to avoid any confusion, the polypeptide sequence corresponding to SEQ ID NO: 1, starting with its N-terminus, is: MLLCTARLVGLQLLISCCWAFACHSTESSPDFTLPGDYLLAG LFPLHSGCLQVRHRPEVTLCDRSCSFNEHGYHLFQAMRLGVEEINNSTALLPNITLGY QLYDVCSDSANVYATLRVLSLPGQHHIELQGDLLHYSPTVLAVIGPDSTNRAATTAA LLSPFLVPMISYAASSETLSVKRQYPSFLRTIPNDKYQVETMVLLLQKFGWTWISLVG SSDDYGQLGVQALENQATGQGICIAFKDIMPFSAQVGDERMQCLMRHLAQAGATV VVVFSSRQLARVFFESVVLTNLTGKVWVASEAWALSRHITGVPGIQRIGMVLGVAIQ KRAVPGLKAFEEAYARADKKAPRPCHKGSWCSSNQLCRECQAFMAHTMPKLKAFS MSSAYNAYRAVYAVAHGLHQLLGCASGACSRGRVYPWQLLEQIHKVHFLLHKDTV AFNDNRDPLSSYNIIAWDWNGPKWTFTVLGSSTWSPVQLNINETKIQWHGKDNQVP KSVCSSDCLEGHQRVVTGFHHCCFECVPCGAGTFLNKSDLYRCQPCGKEEWAPEGS QTCFPRTVVFLALREHTSWVLLAANTLLLLLLLGTAGLFAWHLDTPVVRSAGGRLCF LMLGSLAAGSGSLYGFFGEPTRPACLLRQALFALGFTIFLSCLTVRSFQLIIIFKFSTKV PTFYHAWVQNHGAGLFVMISSAAQLLICLTWLVVWTPLPAREYQRFPHLVMLECTE TNSLGFILAFLYNGLLSISAFACSYLGKDLPENYNEAKCVTFSLLFNFVSWIAFFTTAS VYDGKYLPAANMMAGLSSLSSGFGGYFLPKCYVILCRPDLNSTEHFQASIQDYTRRC GST, using the standard single-letter codes representing amino acids. The sequence listing directed to this sequence is hereby incorporated by reference.

In some embodiments, the disclosure provides a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 2 is set forth among the sequence listings filed herewith and incorporated by reference. For purposes of clarity and to avoid any confusion, the polypeptide sequence corresponding to SEQ ID NO: 2, starting with its N-terminus, is: MLGPAVLGLSLWALLHPGTGAPLCLSQQLRMKGDYVLGGLFPLG EAEEAGLRSRTRPSSPVCTRFSSNGLLWALAMKMAVEEINNKSDLLPGLRLGYDLFD TCSEPVVAMKPSLMFLAKAGSRDIAAYCNYTQYQPRVLAVIGPHSSELAMVTGKFFS FFLMPQVSYGASMELLSARETFPSFFRTVPSDRVQLTAAAELLQEFGWNWVAALGS DDEYGRQGLSIFSALAAARGICIAHEGLVPLPRADDSRLGKVQDVLHQVNQSSVQVV LLFASVHAAHALFNYSISSRLSPKVWVASEAWLTSDLVMGLPGMAQMGTVLGFLQR GAQLHEFPQYVKTHLALATDPAFCSALGEREQGLEEDVVGQRCPQCDCITLQNVSA GLNHHQTFSVYAAVYSVAQALHNTLQCNASGCPAQDPVKPWQLLENMYNLTFHVG GLPLRFDSSGNVDMEYDLKLWVWQGSVPRLHDVGRFNGSLRTERLKIRWHTSDNQ KPVSRCSRQCQEGQVRRVKGFHSCCYDCVDCEAGSYRQNPDDIACTFCGQDEWSPE RSTRCFRRRSRFLAWGEPAVLLLLLLLSLALGLVLAALGLFVHHRDSPLVQASGGPL ACFGLVCLGLVCLSVLLFPGQPSPARCLAQQPLSHLPLTGCLSTLFLQAAEIFVESELP LSWADRLSGCLRGPWAWLVVLLAMLVEVALCTWYLVAFPPEVVTDWHMLPTEAL VHCRTRSWVSFGLAHATNATLAFLCFLGTFLVRSQPGRYNRARGLTFAMLAYFITW VSFVPLLANVQVVLRPAVQMGALLLCVLGILAAFHLPRCYLLMRQPGLNTPEFFLGG GPGDAQGQNDGNTGNQGKHE, using the standard single-letter amino acid codes. The sequence listing directed to this sequence is hereby incorporated by reference.

In some other embodiments, the disclosure provides a polypeptide sequence of SEQ ID NO: 3, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 3 is set forth among the sequence listings filed herewith and incorporated by reference. For purposes of clarity and to avoid any confusion, the polypeptide sequence corresponding to SEQ ID NO: 3, starting with its N-terminus, is: MPGLAILGLSLAAFLELGMGSSLCLSQQFKAQGDYILGGLFPLGT TEEATLNQRTQPNGILCTRFSPLGLFLAMAMKMAVEEINNGSALLPGLRLGYDLFDT CSEPVVTMKPSLMFMAKVGSQSIAAYCNYTQYQPRVLAVIGPHSSELALITGKFFSFF LMPQVSYSASMDRLSDRETFPSFFRTVPSDRVQLQAVVTLLQNFSWNWVAALGSDD DYGREGLSIFSGLANSRGICIAHEGLVPQHDTSGQQLGKVVDVLRQVNQSKVQVVVL FASARAVYSLFSYSILHDLSPKVWVASESWLTSDLVMTLPNIARVGTVLGFLQRGAL LPEFSHYVETRLALAADPTFCASLKAELDLEERVMGPRCSQCDYIMLQNLSSGLMQN LSAGQLHHQIFATYAAVYSVAQALHNTLQCNVSHCHTSEPVQPWQLLENMYNMSF RARDLTLQFDAKGSVDMEYDLKMWVWQSPTPVLHTVGTFNGTLQLQHSKMYWPG NQVPVSQCSRQCKDGQVRRVKGFHSCCYDCVDCKAGSYRKHPDDFTCTPCGKDQW SPEKSTTCLPRRPKFLEWGEPAVLLLLLLLSLALGLVLAALGLFVHHRDSPLVQASGG PLACFGLVCLGLVCLSVLLFPGQPSPARCLAQQPLSHLPLTGCLSTLFLQAAEIFVESE LPLSWADRLSGCLRGPWAWLVVLLAMLVEVALCTWYLVAFPPEVVTDWHMLPTE ALVHCRTRSWVSFGLAHATNATLAFLCFLGTFLVRSQPGRYNRARGLTFAMLAYFIT WVSFVPLLANVQVVLRPAVQMGALLLCVLGILAAFHLPRCYLLMRQPGLNTPEFFL GGGPGDAQGQNDGNTGNQGKHE, using the standard single-letter amino acid codes. The sequence listing directed to this sequence is hereby incorporated by reference.

The polypeptide sequences of the foregoing aspects and embodiments can be present in any suitable composition. In some embodiments, one or more polypeptide sequences of the foregoing aspects and embodiments is present in a non-naturally occurring composition, such as an in vitro assay. In some further such embodiments, the polypeptide sequences of the foregoing aspects and embodiments are expressed on the surface of cells, such as on the cells of a eukaryotic cell line.

### Screening Methods

In certain aspects, the disclosure provides methods of identifying compounds that produce or enhance a kokumi taste sensation, the method comprising: (a) introducing a test compound to a taste receptor, which comprises a first subunit and a second subunit; wherein the first subunit comprises a polypeptide sequence of SEQ ID NO: 1, a functional fragment thereof, or a polypeptide sequence whose sequence is at least 90% equivalent to either of the foregoing; and wherein the second subunit comprises (i) a polypeptide sequence of SEQ ID NO: 2, a functional fragment thereof, or a polypeptide sequence whose sequence is at least 90% equivalent to either of the foregoing, or (ii) a polypeptide sequence of SEQ ID NO: 3, a functional fragment thereof, or a polypeptide sequence whose sequence is at least 90% equivalent to either of the foregoing; and (b) measuring a response of the taste receptor to the test compound by comparing an activity of the taste receptor in the presence and the absence of the test compound. In some embodiments, the methods further comprise: identifying an active test compound that imparts or enhances kokumi taste sensation based on the measured response; and, optionally, selecting the active test compound as a compound that imparts or enhances kokumi taste sensation.

In some other aspects, the disclosure provides methods of identifying compounds that enhance a kokumi taste sensation, the method comprising: (a) introducing a test compound in the presence of a kokumi compound to a taste receptor, which comprises a first subunit and a second subunit; wherein the first subunit comprises a polypeptide sequence of SEQ ID NO: 1, a functional fragment thereof, or a polypeptide sequence whose sequence is at least 90% equivalent to either of the foregoing; and wherein the second subunit comprises (i) a polypeptide sequence of SEQ ID NO: 2, a functional fragment thereof, or a polypeptide sequence whose sequence is at least 90% equivalent to either of the foregoing, or (ii) a polypeptide sequence of SEQ ID NO: 3, a functional fragment thereof, or a polypeptide sequence whose sequence is at least 90% equivalent to either of the foregoing; and (b) measuring a response of the taste receptor to the test compound by comparing an activity of the taste receptor in the presence and the absence of the test compound.

In some embodiments of the foregoing aspects, the introducing step (a) comprises introducing a test compound in the presence of a kokumi compound to a taste receptor in the presence in the presence of a 5′ribonucleotide, for example, to sensitize the assay. In some embodiments, the 5′-ribonucleotide is guanosine 5′-monophosphate (GMP), inosine 5′-monophosphate (IMP), or combinations thereof.

In some embodiments, the second subunit comprises a polypeptide sequence of SEQ ID NO: 2, a functional fragment thereof, or a polypeptide sequence whose sequence is at least 90% equivalent to either of the foregoing. In some other embodiments, the second subunit comprises a polypeptide sequence of SEQ ID NO: 3, a functional fragment thereof, or a polypeptide sequence whose sequence is at least 90% equivalent to either of the foregoing.

In some embodiments, the first subunit of the taste receptor is a polypeptide that comprises a polypeptide sequence of SEQ ID NO: 1, or a functional fragment thereof, or a polypeptide sequence whose sequence is at least 90% equivalent, or at least 95% equivalent, or at least 97% equivalent, to either of the foregoing. In some further embodiments, the first subunit is a polypeptide that comprises: a polypeptide sequence of SEQ ID NO: 1, or a polypeptide sequence whose sequence is at least 90% equivalent, or at least 95% equivalent, or at least 97% equivalent, thereto. The polypeptide of SEQ ID NO: 1 can be referred to in certain instances as a “T1R1” taste receptor.

In some embodiments, the second subunit of the taste receptor is a polypeptide that comprises a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. In some further embodiments, the second subunit is a polypeptide that comprises: a polypeptide sequence of SEQ ID NO: 2, or a polypeptide sequence whose sequence is at least 90% equivalent, or at least 95% equivalent, or at least 97% equivalent, thereto. The polypeptide of SEQ ID NO: 2 can be referred to in certain instances as a “T1R3” taste receptor.

In some embodiments, the second subunit of the taste receptor is a polypeptide that comprises a polypeptide sequence of SEQ ID NO: 3, or a functional fragment thereof, or a polypeptide sequence whose sequence is at least 90% equivalent, or at least 95% equivalent, or at least 97% equivalent, to either of the foregoing. In some further embodiments, the second subunit is a polypeptide that comprises: a polypeptide sequence of SEQ ID NO: 3, or a polypeptide sequence whose sequence is at least 90% equivalent, or at least 95% equivalent, or at least 97% equivalent, thereto. The polypeptide of SEQ ID NO: 3 can also be referred to in certain instances as a “T1R3” taste receptor.

The initial introducing step (a) can be carried out in any suitable way. For example, in some non-limiting embodiments, when introducing a test compound to taste receptor, the introducing can be carried out in a single cell-based assay. Any suitable assay can be used, according to the knowledge of those skilled in the art.

In some embodiments, it can be desirable to measure the activity of the test compound in the presence of one or more additional compounds that are known to imparts kokumi taste sensation. In this way, one can determine whether the test compound enhances or blocks (antagonizes) the binding of the one or more additional compounds. In some embodiments, the one or more additional compounds comprise a kokumi compound. Thus, in some embodiments, the introducing step (a) comprises introducing the test compound to the taste receptor in the presence of a kokumi compound, such as a compound that imparts a kokumi taste sensation.

In such embodiments, any suitable kokumi compounds can be used. Non-limiting examples include gamma-glutamyl peptides, such as gamma-glutamyl dipeptides and gamma-glutamyl tripeptides. Nonlimiting examples include glutathione, Glu-Glu-Leu, and Glu-Val-Gly.

The foregoing embodiments involve measuring a response of the taste receptor to the test compound. This measuring can be carried out by any suitable means. For example, in some embodiments, the taste receptor is expressed on the surface of cells, and compositions are screened against the cells expressing the taste receptor in a standard cellular assay. Measuring binding to the taste receptor can be carried out by any suitable means typically used in determining protein binding in cellular assays. Suitable methods include, but are not limited to, use of fluorescent dyes, a calcium indicator protein, a fluorescent calcium indicator, a fluorescent cAMP indicator, and the like. In some embodiments, activity of the test compound is determined by its binding to the taste receptor. In some embodiments, activity of the test compound is determined by its ability to enhance the binding of a kokumi compound to the taste receptor

In some further embodiments of any of the foregoing embodiments, the methods further comprise identifying an active test compound that imparts or enhances kokumi taste based on the measured response. According to the foregoing embodiments, the identified active test compound is one that modulates the taste receptor or agonizes its modulation by a kokumi compound.

In some embodiments of any of the foregoing embodiments, the test compound is determined to be an active compound because it modulates the taste receptor, for example, because it induces a response of the taste receptor having an EC50 of no more than 10 mM, or no more than 1 mM or no more than 500 μM, or no more than 250 μM, or no more than 100 μM, and because the test compound is not active on cells not expressing T1R1 and T1R3

In some embodiments of any of the foregoing embodiments, the test compound is determined to be an active compound because it modulates the activity of a kokumi compound to the taste receptor, for example, because it induces a response of the taste receptor having an EC50 of no more than 10 mM, or no more than, no more than 1 mM, no more than 500 μM, or no more than 250 μM, or no more than 100 μM in the presence of a kokumi compound

In some further embodiments, the methods further comprise selecting the active test compound as a compound that imparts or enhances kokumi taste sensation. The selecting step can be carried out by any suitable means once the active test compounds are identified.

The test compound can be any suitable compound that is amenable for use in cellular screening assays. For example, in some embodiments, the test compound is an organic compound. In some embodiments, the test compound is a naturally occurring compound. In some other embodiments, the test compound is a non-naturally occurring compound. In some embodiments, the test compound is a peptide, such as a polypeptide or an oligopeptide, an amino acid, an amino acid derivative, an amide, a nucleotide, an oligonucleotide. In some embodiments, the test compound is a plant or food extract, such as a plant or food extract obtained by bioassay-guided fractionation. In some embodiments, the test compound is a diterpene or a triterpene. In some embodiments, the test compound is a ribonucleotide or derivative thereof.

### Uses and Methods

In other aspects, the disclosure provides uses of any identified or selected active compounds of the foregoing aspects, including any embodiments or combination of embodiments thereof, as set forth above. In certain related aspects, the disclosure provides uses of any identified or selected active compounds of the foregoing aspects, including any embodiments or combination of embodiments thereof, as set forth above, to impart a kokumi taste sensation or enhance a kokumi taste sensation of an ingestible composition.

The disclosure also provides methods that correspond to each of the foregoing uses. Thus, in certain related aspects, the disclosure provides methods of imparting a kokumi taste sensation or enhancing a kokumi taste sensation in an ingestible composition, comprising introducing an amount (such as a kokumi-imparting amount or an kokumi-enhancing amount) of any identified or selected active compounds of foregoing aspects, including any embodiments or combination of embodiments thereof, as set forth above, to the ingestible composition.

The foregoing uses and methods generally involve the use of the identified or selected active compounds in a composition containing one or more additional ingredients. For example, in at least one aspect, the disclosure provides compositions comprising any identified or selected active compounds of the foregoing aspects, including any embodiments or combination of embodiments thereof, as set forth above, wherein the identified or selected active compounds make up at least 10% by weight, or at least 20% by weight, or at least 30% by weight, or at least 40% by weight, or at least 50% by weight, of the compositions on a dry weight basis (e.g., based on the total weight of the composition excluding the weight of any liquid carrier). In a related aspect, the disclosure provides solid-state compositions comprising any identified or selected active compounds of the foregoing aspects, including any embodiments or combination of embodiments thereof, as set forth above, wherein the identified or selected active compounds make up at least 10% by weight, or at least 20% by weight, or at least 30% by weight, or at least 40% by weight, or at least 50% by weight, of the solid-state compositions, based on the total weight of composition. In another related aspect, the disclosure provides ingestible compositions comprising identified or selected active compounds of the foregoing aspects, including any embodiments or combination of embodiments thereof, as set forth above, wherein the concentration of the identified or selected active compounds in the ingestible compositions is no more than 2500 ppm (such as from 1 ppm to 2500 ppm), or no more than 2000 ppm (such as from 1 ppm to 2000 ppm), or no more than 1500 ppm (such as from 1 ppm to 1500 ppm), or no more than 1000 ppm (such as from 1 ppm to 1000 ppm), or no more than 750 ppm (such as from 1 ppm to 750 ppm), or no more than 500 ppm (such as from 1 ppm to 500 ppm), or no more than 400 ppm (such as from 1 ppm to 400 ppm), or no more than 300 ppm (such as from 1 ppm to 300 ppm), or no more than 200 ppm (such as from 1 ppm to 200 ppm), or no more than 150 ppm (such as from 1 ppm to 150 ppm), or no more than 100 ppm (such as from 1 ppm to 100 ppm), or no more than 50 ppm (such as from 1 ppm to 50 ppm), or no more than 30 ppm (such as from 1 ppm to 30 ppm), or no more than 20 ppm (such as from 1 ppm to 20 ppm),

In certain embodiments of any aspects and embodiments set forth herein that refer to an ingestible composition, the ingestible composition is a non-naturally-occurring product or a naturally-occurring product, such as a composition specifically manufactured, extracted or purified for the production of a flavored product, such as food or beverage product.

In certain aspects, the disclosure provides ingestible compositions comprising one or more umami compounds. In such embodiments, any suitable umami compounds can be used. Non-limiting examples include: L-glutamic acid and related salts such as monosodium glutamate (MSG), potassium glutamate, and calcium glutamate; L-aspartic acid; and related salts, such as sodium aspartate or potassium aspartate; and 5′-ribonucleotides, such as purinic ribonucleotides like guanosine 5′-monophosphate (GMP) and inosine 5′-monophosphate (IMP); and other known umami compounds or umami enhancers, such as FEMA 4232, FEMA 4233, FEMA 4877, FEMA 4798, and the like.

In certain aspects, the disclosure provides ingestible compositions comprising one or more kokumi compounds. In such embodiments, any suitable kokumi compounds can be used. Non-limiting examples gamma-glutamyl peptides, such as gamma-glutamyl dipeptides and gamma-glutamyl tripeptides. Nonlimiting examples include glutathione, Glu-Glu-Leu, Glu-Val-Gly, protein hydrolysates, such as protein hydrolysates enriched in umami compounds, and the like.

In the preceding aspect, or in any other aspect that refers to an ingestible composition, the active compound can be present in the ingestible composition in any suitable concentration. For example, in some embodiments, the active compound is present in the ingestible composition at a concentration ranging from 1 ppm to 2500 ppm, from 1 ppm to 2000 ppm, or from 1 ppm to 1500 ppm, or from 1 ppm to 1000 ppm, or from 1 ppm to 900 ppm, or from 1 ppm to 800 ppm, or from 1 ppm to 700 ppm, or from 1 ppm to 600 ppm, or from 1 ppm to 500 ppm, or from 1 ppm to 400 ppm, or from 1 ppm to 300 ppm, or from 1 ppm to 250 ppm, or from 1 ppm to 200 ppm, or from 1 ppm to 150 ppm, or from 1 ppm to 100 ppm, or from 1 ppm to 80 ppm, or from 1 ppm to 70 ppm, or from 1 ppm to 60 ppm, or from 1 ppm to 50 ppm, or from 1 ppm to 40 ppm, or from 1 ppm to 30 ppm, or from 1 ppm to 20 ppm, or from 5 ppm to 1000 ppm, or from 5 ppm to 900 ppm, or from 5 ppm to 800 ppm, or from 5 ppm to 700 ppm, or from 5 ppm to 600 ppm, or from 5 ppm to 500 ppm, or from 5 ppm to 400 ppm, or from 5 ppm to 300 ppm, or from 5 ppm to 250 ppm, or from 5 ppm to 200 ppm, or from 5 ppm to 150 ppm, or from 5 ppm to 100 ppm, or from 5 ppm to 80 ppm, or from 5 ppm to 70 ppm, or from 5 ppm to 60 ppm, or from 5 ppm to 50 ppm, or from 5 ppm to 40 ppm, or from 5 ppm to 30 ppm, or from 5 ppm to 20 ppm.

In general, compounds as disclosed and described herein, individually or in combination, can be provided in a composition, such as an ingestible composition. In one embodiment, compounds as disclosed and described herein, individually or in combination, can impart a more kokumi-like temporal profile or flavor profile to a composition by combining one or more of the compounds as disclosed and described herein with one or more umami compounds, kokumi compounds, sweeteners, and the like. In another embodiment, compounds as disclosed and described herein, individually or in combination, can increase or enhance the sweet taste of a composition by contacting the composition thereof with the compounds as disclosed and described herein to form a modified composition.

Thus, in some embodiments, the compositions set forth in any of the foregoing aspects (including in any uses or methods), comprise an identified or selected active compound and an umami compound, a kokumi compound, or any combination thereof. In some embodiments, the composition further comprises a vehicle. In some embodiments, the vehicle is water. In some embodiments, the identified or selected active compound is present at a concentration at or below its sweetness recognition threshold.

In certain particular embodiments, the ingestible composition comprises monosodium glutamate and the active compound (or comestibly acceptable salts thereof). In some such embodiments, the introduction of the active compound (or comestibly acceptable salt thereof) permits one to use less monosodium glutamate (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of monosodium glutamate. In some related embodiments, the use of the active compound, or its comestibly acceptable salts, permits the elimination of monosodium glutamate from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain particular embodiments, the ingestible composition comprises fat, such as animal or vegetable fat, and the active compound (or comestibly acceptable salts thereof). In some such embodiments, the introduction of the active compound (or comestibly acceptable salt thereof) permits one to use less fat (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of fat. In some related embodiments, the use of the active compound, or its comestibly acceptable salts, permits the elimination of fat from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The fat can be any suitable fat, such as a fat derived from an animal or vegetable fat, including, but not limited to, milk fat (including fat in various cheeses), beef fat, pork fat, poultry fat, lamb fat, goat fat, fish oil, olive oil, canola oil, corn oil, safflower oil, nut oil, peanut oil, cashew oil, soybean oil, palm oil, palm kernel oil, coconut oil, butter, and nut butters (such as peanut butter, cashew butter, almond butter, hazelnut butter, and the like).

In certain particular embodiments, the ingestible composition comprises glutamate (including in its free acid form), and the active compound (or comestibly acceptable salts thereof). In some such embodiments, the introduction of the active compound (or comestibly acceptable salt thereof) permits one to use less glutamate (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of glutamate. In some related embodiments, the use of the active compound, or its comestibly acceptable salts, permits the elimination of glutamate from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The glutamate can be from any suitable source, such as monosodium glutamate (MSG), proteins containing glutamic acid (e.g., glutathione), potassium glutamate, calcium glutamate, and the like.

In certain particular embodiments, the ingestible composition comprises aspartate (including in its free acid form), and the active compound (or comestibly acceptable salts thereof). In some such embodiments, the introduction of the active compound (or comestibly acceptable salt thereof) permits one to use less aspartate (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of aspartate. In some related embodiments, the use of the active compound, or its comestibly acceptable salts, permits the elimination of aspartate from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The aspartate can be from any suitable source, such as proteins containing aspartic acid, and the like.

In certain particular embodiments, the ingestible composition comprises animal products, and the active compound (or comestibly acceptable salts thereof). In some such embodiments, the introduction of the active compound (or comestibly acceptable salt thereof) permits one to use less animal products (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of animal products. In some related embodiments, the use of the active compound, or its comestibly acceptable salts, permits the elimination of animal products from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The animal products can be any suitable animal product, such as cheese, milk, meat broth (such as beef broth, pork broth, chicken broth, turkey broth, duck broth, lamb broth, goat broth, rabbit broth, and the like), eggs, bone broth, bone marrow, meat (such as beef, pork, chicken, lamb, goat, turkey, duck, rabbit, and the like), butter, and animal skin.

In certain particular embodiments, the ingestible composition comprises vegetable products, and the active compound (or comestibly acceptable salts thereof). In some such embodiments, the introduction of the active compound (or comestibly acceptable salt thereof) permits one to use less vegetable products (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of vegetable products. In some related embodiments, the use of the active compound, or its comestibly acceptable salts, permits the elimination of vegetable products from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The vegetable products can be any suitable vegetable product, such as celery, celeriac, tomato, garlic, onion, leek, scallion, spices, and the like.

In certain particular embodiments, the ingestible composition comprises fungal products, and the active compound (or comestibly acceptable salts thereof). In some such embodiments, the introduction of the active compound (or comestibly acceptable salt thereof) permits one to use less fungal products (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of vegetable products. In some related embodiments, the use of the active compound, or its comestibly acceptable salts, permits the elimination of vegetable products from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The vegetable products can be any suitable vegetable product, such as celery, celeriac, tomato, garlic, onion, leek, scallion, spices, and the like.

In certain particular embodiments, the ingestible composition comprises sodium (i.e., sodium cation), and the active compound (or comestibly acceptable salts thereof). In some such embodiments, the introduction of the active compound (or comestibly acceptable salt thereof) permits one to use less sodium (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of sodium. In some related embodiments, the use of the active compound, or its comestibly acceptable salts, permits the elimination of sodium from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The sodium can be any suitable animal product, such as table salt, sea salt, soy sauce, fish sauce, shrimp paste, butter, miso, and Worcestershire sauce.

In certain particular embodiments, the ingestible composition comprises alcohol, and the active compound (or comestibly acceptable salts thereof). In some such embodiments, the introduction of the active compound (or comestibly acceptable salt thereof) permits one to use less alcohol (such as more than 10% less, more than 20% less, more than 30% less, more than 40% less, more than 50% less, more than 60% less, or more than 70% less, or more than 80% less, or more than 90% less) and still achieve a level of umami and/or kokumi characteristic of a comparable product that employs a higher concentration of alcohol. In some related embodiments, the use of the active compound, or its comestibly acceptable salts, permits the elimination of alcohol from the composition. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda (such as a hard soda), and the like. The alcohol can present in any suitable form, such as alcohol formed from grains, cane sugar, fruits, and the like.

In some instances, one may be able to reduce the amount of sweetener in a product by enhancing the umami or kokumi taste.

In certain particular embodiments, the ingestible composition comprises sucrose and the active compound or any of its comestibly acceptable salts. In some such embodiments, the introduction of the active compound (or salt) permits one to use less sucrose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more sucrose. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises fructose and the active compound or any of its comestibly acceptable salts. In some such embodiments, the introduction of the active compound (or salt) permits one to use less fructose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more fructose. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises high-fructose corn syrup and the active compound or any of its comestibly acceptable salts. In some such embodiments, the introduction of the active compound (or salt) permits one to use less high-fructose corn syrup (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more high-fructose corn syrup. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like.

In certain particular embodiments, the ingestible composition comprises glucose (for example, D-glucose, in either its alpha or beta forms, or a combination thereof) and the active compound or any of its comestibly acceptable salts. In some such embodiments, the introduction of the active compound (or salt) permits one to use less glucose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more glucose. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The glucose can be introduced in any suitable form, such as natural syrups and the like.

In certain particular embodiments, the ingestible composition comprises sucralose and the active compound or any of its comestibly acceptable salts. In some such embodiments, the introduction of the active compound (or salt) permits one to use less sucralose (such as more than 10% less, more than 20% less, more than 30% less, more than 40% less, more than 50% less, more than 60% less, or more than 70% less) and still achieve a level of sweetness, umami, and/or kokumi characteristic of a comparable product that employs more sucralose. In some embodiments, the concentration of the active compound, or its comestibly acceptable salts, is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In general, compounds as disclosed and described herein, individually or in combination, can be provided in a composition, such as an ingestible composition. In one embodiment, compounds as disclosed and described herein, individually or in combination, can impart a more kokumi-like temporal profile or flavor profile to a flavoring composition by combining one or more of the compounds as disclosed and described herein with one or more kokumi or umami compounds in the ingestible composition. In another embodiment, compounds as disclosed and described herein, individually or in combination, can increase or enhance the kokumi taste of a composition by contacting the composition thereof with the compounds as disclosed and described herein to form a modified composition.

The ingestible compositions can, in certain embodiments, comprise any additional ingredients or combination of ingredients as are commonly used in food and beverage products, including, but not limited to:

acids, including, for example citric acid, phosphoric acid, ascorbic acid, sodium acid sulfate, lactic acid, or tartaric acid;

bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);

coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;

preservatives, including, for example sodium benzoate, potassium benzoate, potassium sorbate, sodium metabisulfate, sorbic acid, or benzoic acid;

antioxidants including, for example ascorbic acid, calcium disodium EDTA, alpha tocopherols, mixed tocopherols, rosemary extract, grape seed extract, resveratrol, or sodium hexametaphosphate;

vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;

clouding agents, including, for example ester gun, brominated vegetable oil (BVO), or sucrose acetate isobutyrate (SAIB);

buffers, including, for example sodium citrate, potassium citrate, or salt;

flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or

starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions can have any suitable pH. In some embodiments, the flavor-modifying compounds enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

The ingestible compositions set forth according to any of the foregoing embodiments, also include, in certain embodiments, one or more additional flavor-modifying compounds, such as compounds that enhance sweetness (e.g., hesperetin, naringin, naringenin, glucosylated steviol glycosides, etc.), compounds that block bitterness, compounds that enhance umami, compounds that reduce sourness or licorice taste, compounds that enhance saltiness, compounds that enhance a cooling effect, or any combinations of the foregoing.

Thus, in some embodiments, ingestible compositions disclosed herein comprise the active compound, or any comestibly acceptable salts thereof, according to any of the embodiments or combination of embodiments set forth above, are combined with one or more sweetness enhancing compounds. Such sweetness enhancing compounds include, but are not limited to, naturally derived compounds, such as hesperitin, naringenin, glucosylated steviol glycosides, or synthetic compounds, such as any compounds set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032. The active compound (or comestibly acceptable salts thereof) may be used in combination with such other sweetness enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments of any of the preceding embodiments, the active compound (or any comestibly acceptable salts thereof) is combined with glucosylated steviol glycosides in any of the above ratios. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1.6 bond, a β-1,2 bond, a β-1,4 bond, a β-1,6 bond, and so forth. In some embodiments of any of the preceding embodiments, the active compound (or any comestibly acceptable salts thereof) is combined with 3-((4-amino-2,2-dioxo-1H-benzo[c][1,2,6]thiadiazin-5-yl)oxy)-2,2-dimethyl-N-propyl-propanamide, N-(1-((4-amino-2,2-dioxo-1H-benzo[c][1,2,6]thiadiazin-5-yl)oxy)-2-methyl-propan-2-yl)isonicotinamide, or any combination thereof, in any of the above ratios.

In some further embodiments, ingestible compositions disclosed herein comprise the active compound, or any comestibly acceptable salts thereof, according to any of the embodiments or combination of embodiments set forth above, are combined with one or more other umami or kokumi enhancing compounds. Such umami enhancing compounds include, but are not limited to, naturally derived compounds, such as FEMA 4232, FEMA 4233, FEMA 4798, or FEMA 4877, or synthetic compounds, such as any compounds set forth in U.S. Pat. Nos. 8,735,081; 8,124,121; and 8,968,708. The active compound (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the active compound, or any comestibly acceptable salts thereof, according to any of the embodiments or combination of embodiments set forth above, are combined with one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. The active compound (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the active compound, or any comestibly acceptable salts thereof, according to any of the embodiments or combination of embodiments set forth above, are combined with one or more bitterness blocking compounds. Such bitterness blocking compounds include, but are not limited to, naturally derived compounds, such as menthol or analogs thereof, or synthetic compounds, such as any compounds set forth in U.S. Pat. Nos. 8,076,491; 8,445,692; and 9,247,759. The active compound (or comestibly acceptable salts thereof) may be used in combination with such bitterness blockers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the active compound, or any comestibly acceptable salts thereof, according to any of the embodiments or combination of embodiments set forth above, are combined with one or more sour taste modulating compounds. The active compound (or comestibly acceptable salts thereof) may be used in combination with such sour taste modulating compounds in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the active compound, or any comestibly acceptable salts thereof, according to any of the embodiments or combination of embodiments set forth above, are combined with one or more mouthfeel modifying compounds. Such mouthfeel modifying compounds include, but are not limited to, tannins, cellulosic materials, bamboo powder, and the like. The active compound (or comestibly acceptable salts thereof) may be used in combination with such mouthfeel enhancers in any suitable ratio (w/w) ranging from 1:1000 to 1000:1, or from 1:100 to 100:1, or fro, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1,20:1,21:1,22:1,23:1,24:1, or 25:1.

In some further embodiments, ingestible compositions disclosed herein comprise the active compound, or any comestibly acceptable salts thereof, according to any of the embodiments or combination of embodiments set forth above, are combined with one or more flavor masking compounds. Such flavor masking compounds include, but are not limited to, cellulosic materials, materials extracted from fungus, materials extracted from plants, citric acid, carbonic acid (or carbonates), and the like. The active compound (or comestibly acceptable salts thereof) may be used in combination with such mouthfeel enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some aspects related to the preceding aspects and embodiments, the disclosure provides uses of the active compound (or comestibly acceptable salts thereof) to enhance the flavor of a flavored composition, such as a flavored article. Such flavored compositions can use any suitable flavors, such as fruit flavors, meat flavors, vegetable flavors, and the like. In some embodiments, the flavored composition is a soup or broth, or a chip, or a beverage.

### Flavored Products and Savory Food Products

In certain aspects, the disclosure provides flavored products comprising any compositions of the preceding aspects. In some embodiments, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to a flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

Typically at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of sweeteners so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

In some embodiments, compounds as disclosed and described herein, individually or in combination, modulate the, umami taste, kokumi taste or other taste properties of other natural or synthetic sweet tastants, and ingestible compositions made therefrom. In one embodiment, the compounds as disclosed and described herein, individually or in combination, may be used or provided in its ligand enhancing concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some embodiments, flavor-modifying compounds as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that enhance kokumi, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

The sweetening or flavoring concentrates set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness (e.g., eriodictyol, homoeriodictyol, sterubin, and salts or glycoside derivatives thereof, as well as vanillyl lignans, e.g., matairesinol and other compounds set forth in PCT Publication No. WO 2012/146584), compounds that enhance umami (e.g., rubemamine, rubescenamine, (E)-3-(3,4-dimethoxyphenyl)-N-(4-methoxyphenethyl)acrylamide, and the like), FEMA 4232, FEMA 4233, FEMA 4798, compounds that reduce sourness and/or licorice taste, compounds that enhance saltiness, compounds that enhance a cooling effect, or any combinations of the foregoing.

### Non-Animal Protein Materials and Products Made Therefrom

Products intended to replace or substitute meat or dairy products often rely on various non-animal-based materials, such as starches and proteins derived from plants, algae, and fungi, to simulate the texture and flavor of meat or dairy. Non-limiting examples of such plant proteins include soy proteins, pea proteins, bean proteins, grain proteins, and the like. Due to compositional differences between such plant-based materials and animal-derived materials, such as a lack of glutamate-containing proteins and glutathione, these products can lack the umami and/or kokumi taste that consumers traditionally associate with meat or dairy products.

Thus, in certain aspects, the disclosure provides a flavored product comprising a plant-based material (such as a plant-based starch, a plant-based protein, or a combination thereof) and the active compound, or a comestibly acceptable salt thereof. In some further embodiments, the flavored product can include any features of combination of features set forth above for ingestible compositions that contain the active compound, or a comestibly acceptable salt thereof. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a protein powder, a meal-replacement powder, a plant-based creamer for coffee or tea, and the like. In certain further embodiments, any such products contain additional ingredients, and have additional features, as are typically used in the preparation and/or manufacture of such products. For example, such the active compound, or comestibly acceptable salts thereof, may be combined with other flavors and taste modifiers, and may even be encapsulated in certain materials, according to known technologies in the relevant art. Suitable concentrations of the active compound, or comestibly acceptable salts thereof, are set forth above.

In some further embodiments analogous to the above embodiments, proteins or starches from algal or fungal sources can be used instead of or in combination with plant starches or proteins.

### Non-Meat Protein Materials and Products Made Therefrom

Certain non-meat animal proteins, such as dairy proteins and proteins from bone broth, are commonly used in food products, and are also sold as the primary ingredient in certain protein powders. Such proteins can impart flavors that lack the full umami or kokumi taste that consumers may desire. This is especially true for protein isolates, such as protein isolates of whey protein, collagen protein, casein proteins, and the like. Thus, the present disclosure provides ingestible compositions that include non-meat animal proteins and the active compound, or a comestibly acceptable salt thereof. The active compound, or its comestibly acceptable salts, can be present in any suitable combination, according to the embodiments set forth in the preceding sections of the present disclosure. In some embodiments, the non-meat animal protein is a bone protein, such as a collagen protein derived from the bones of an animal, such as a cow, pig, donkey, horse, chicken, duck, goat, goose, rabbit, lamb, sheep, buffalo, ostrich, camel, and the like. In some embodiments, the non-meat animal protein is a milk protein, such as a whey protein, a casein protein, or any combination thereof. The milk can be the milk of any suitable animal, such as a cow, donkey, horse, sheep, buffalo, camel, and the like.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1—Assays

To implement the identification of kokumi modulators, cells transiently or stably expressing a taste receptor having a T1R1 subunit and a T1R3 subunit are used, and binding is detected by measuring changes in Ca²⁺ concentration.

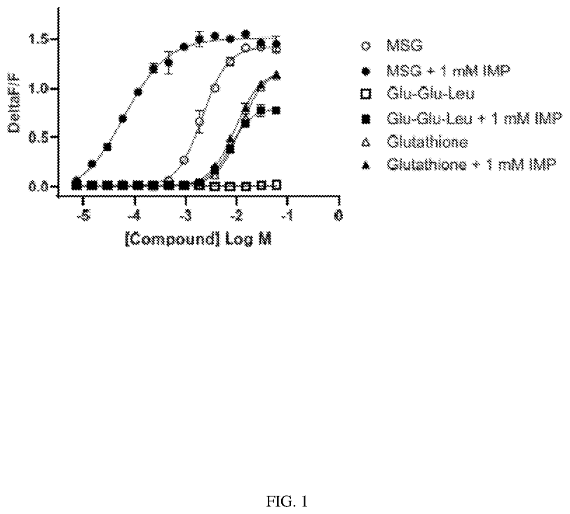

Several gamma-glutamyl peptides were tested in the T1R1/T1R3 assay, both alone or in combination with inosine monophosphate (IMP). Monosodium glutamate (MSG) was used as a positive control. FIG. 1 shows the binding curve for each tested compound or combination of compounds.

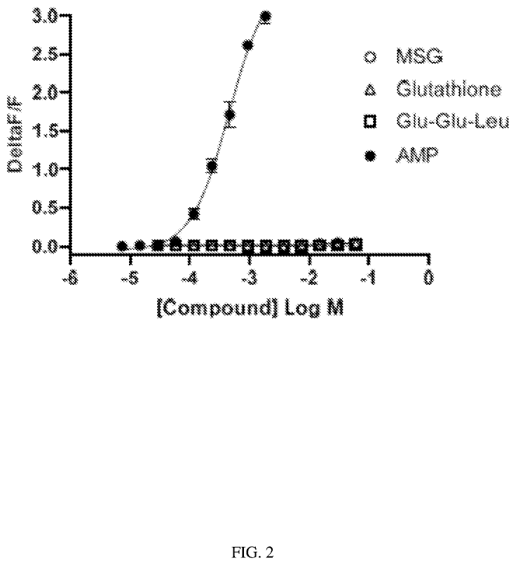

Certain kokumi compounds were also screened using cells not expressing the T1R1/T1R3 taste receptor, with AMP used as a control. FIG. 2 shows the binding curve for each tested compound or combination of compounds.

1. A method of identifying compounds that produce a kokumi taste sensation, the method comprising:
(a) introducing a test compound to a taste receptor, which comprises a first subunit and a second subunit;
wherein the first subunit comprises a polypeptide sequence of SEQ ID NO: 1, a functional fragment thereof, or a polypeptide sequence whose sequence is at least 90% equivalent to either of the foregoing; and
wherein the second subunit comprises (i) a polypeptide sequence of SEQ ID NO: 2, a functional fragment thereof, or a polypeptide sequence whose sequence is at least 90% equivalent to either of the foregoing, or (ii) a polypeptide sequence of SEQ ID NO: 3, a functional fragment thereof, or a polypeptide sequence whose sequence is at least 90% equivalent to either of the foregoing; and
(b) measuring a response of the taste receptor to the test compound by comparing an activity of the taste receptor in the presence and the absence of the test compound.

2. A method of identifying compounds that enhance a kokumi taste sensation, the method comprising:
(a) introducing a test compound in the presence of a kokumi compound to a taste receptor, which comprises a first subunit and a second subunit; wherein the first subunit comprises a polypeptide sequence of SEQ ID NO: 1, a functional fragment thereof, or a polypeptide sequence whose sequence is at least 90% equivalent to either of the foregoing; and wherein the second subunit comprises (i) a polypeptide sequence of SEQ ID NO: 2, a functional fragment thereof, or a polypeptide sequence whose sequence is at least 90% equivalent to either of the foregoing, or (ii) a polypeptide sequence of SEQ ID NO: 3, a functional fragment thereof, or a polypeptide sequence whose sequence is at least 90% equivalent to either of the foregoing; and
(b) measuring a response of the taste receptor to the test compound by comparing an activity of the taste receptor in the presence and the absence of the test compound.

3. The method of claim 1 further comprising: (c) identifying an active test compound that produces or enhances kokumi taste sensation on the measured response.

4. The method of claim 3, further comprising (d) selecting the active test compound as a compound that produces or enhances kokumi taste sensation.

5. The method of claim 1, wherein the introducing step (a) comprises introducing the test compound to the taste receptor in the presence of a kokumi compound, such as a compound that imparts or enhances kokumi taste sensation.

6. The method of claim 5, wherein the kokumi compound is a gamma-glutamyl peptide, such as a gamma-glutamyl dipeptide, a gamma-glutamyl tripeptide, or any combinations thereof, for example, glutathione, Glu-Glu-Leu, Glu-Val-Gly, and the like.

7. The method of claim 1, wherein the test compound is a naturally occurring compound.

8. The method of claim 1, wherein the test compound is a non-naturally occurring compound.

9. A method of imparting or enhancing kokumi taste sensation, comprising ingesting a taste-modifying compound, wherein the compound is the identified or selected compound of claim 3.

10. The method of claim 9, wherein the taste-modifying compound is a naturally occurring compound,

11. The method of claim 9, wherein the taste-modifying compound is a non-naturally occurring compound.

12. A method of imparting or enhancing a kokumi taste sensation in an ingestible composition, comprising including in the ingestible composition a flavor-modifying composition comprising the identified or selected compound of claim 3.

13. The method of claim 12, wherein the flavor-modifying composition further comprises a bitter blocking compound, an umami enhancer, a mouthfeel enhancer, a sweetener, a sweetness enhancer, or any combination thereof.

14. The method of claim 12, wherein the ingestible composition comprises a plant-based material, such as a plant-based starch, a plant-based protein, or any combination thereof; yeast extracts or fermented food products; cheeses; garlic or extracts thereof; a polypeptide or an oligopeptide; an amino acid; an amino acid derivative; an amide; a nucleotide; an oligonucleotide; or plant or food extract, such as a plant or food extract obtained by bioassay-guided fractionation.

15. The method of claim 14, wherein the plant-based material comprises a plant-based protein.

16. The method of claim 15, wherein the plant-based protein makes up at least 20% by weight, or at least 30% by weight, or at least 40% by weight, or at least 50% by weight, or at least 60% by weight, or at least 70% by weight, of the ingestible composition, on a dry weight basis.

17. The method of claim 14, wherein the plant-based protein is soy protein, chickpea protein, canola protein, pea protein, bean protein, or any combination thereof.

18. The method of claim 12, wherein the ingestible composition is a food product, such as a plant-based meat analogue product.

19. A method of (a) reducing an amount of monosodium glutamate in an ingestible composition, (b) reducing an amount of sodium in an ingestible composition, (c) reducing an amount of fat in an ingestible composition, (d) reducing an amount of animal products in an ingestible composition, or (e) reducing an amount of alcohol in an ingestible composition, comprising including a flavor-modifying compound in the ingestible composition, wherein the flavor-modifying compound is the identified or selected compound of claim 3.

20. The method of claim 19, wherein the flavor-modifying compound reduces the amount of monodosium glutamate, sodium, fat, animal products, or alcohol by at least 20%, or at least 25%, or at least 30%, or by at least 40%, or by at least 50%, or at least 60%, or at least 70%, or at least 80%, or at least 90%.

21. The method of claim 19, wherein the ingestible composition further comprises a bitter blocking compound, an umami enhancer, a mouthfeel enhancer, a sweetener, a sweetness enhancer, or any combination thereof.

22. The method of claim 19, wherein the ingestible composition comprises a plant-based material, wherein the plant-based material comprises a plant-based starch, a plant-based protein, or any combination thereof.

23. The method of claim 22, wherein the plant-based material comprises a plant-based protein.

24. The method of claim 23, wherein the plant-based protein makes up at least 20% by weight, or at least 30% by weight, or at least 40% by weight, or at least 50% by weight, or at least 60% by weight, or at least 70% by weight, of the ingestible composition, on a dry weight basis.

25. The method of claim 22, wherein the plant-based protein is soy protein, chickpea protein, canola protein, pea protein, bean protein, or any combination thereof.

26. The method of claim 19, wherein the ingestible composition is a food product, such as a plant-based meat analogue product.
